(19) United States
(12) Patent Application Publication
(10) Pub. No.: US 2026/0307070 A1
(43) Pub. Date: Oct. 8, 2026

(54) CUSTOMIZABLE TESTING CARTRIDGES AND READER BOX FOR NUCLEIC ACID AMPLIFICATION ASSAYS

(71) Applicant: Omnibox, Inc., San Diego, CA (US)

(72) Inventor: George Benjamin Schwartz, San Diego, CA (US)

(21) Appl. No.: 19/479,942

(22) PCT Filed: May 9, 2024
(86) PCT No.: PCT/US2024/028627

§ 371 Date: Oct. 29, 2025

Related U.S. Application Data
(60) Provisional application No. 63/466,099, filed on May 12, 2023.

(51) Int. Cl. B01L 3/00; B01L 7/00

(52) U.S. Cl. CPC B01L 3/502; B01L 7/52; B01L 2200/026; B01L 2200/12; B01L 2200/16; B01L 2400/0487

(57) ABSTRACT

A testing cartridge and a reader box are provided. The testing cartridge can have multiple fluidic chambers connected by fluid tubes; the fluidic chambers being used to perform different parts of NAA testing. The testing cartridge has external interfaces, e.g., a snap on feature, for a user and/or the reader box to insert a chemical reagent. The reader box has dispensing structures to dispense the chemical reagents into the testing cartridge. Such flexibility allows the testing cartridges to be manufactured as blanks and the reagents can be added in situ for the tests. In some embodiments, the testing cartridges can be manufactured having just the primers and the probes, and other reagents can be added, either by the user and/or the reading box, during the testing. The cartridges can be easily manufactured, are generally cheaper, and are significantly more convenient as compared to the existing NAA testing equipment.

### CROSS-REFERENCE TO RELATED APPLICATIONS

This application claims the benefit of priority under 35 U.S.C. § 119(e) of U.S. Provisional Application No. 63/466,099, filed May 12, 2023. The disclosure of the prior application is considered part of and is herein incorporated by reference in the disclosure of this application in its entirety.

### FIELD OF THE INVENTION

This disclosure relates to customizable testing cartridges and a reader box for Nucleic Acid Amplification (NAA) assays.

### BACKGROUND INFORMATION

NAA testing is a widely used technique to detect genetic materials from a variety of sources. For instance, NAA testing is used on a biological specimen, such as a nasal cavity or throat swab, to detect whether genetic materials of a pathogen are present. The recent COVID-19 pandemic highlighted the importance of diagnostic NAA testing, which is known to be significantly reliable as compared to rapid antigen testing (commonly available in-home test kits and showing positive result only when there is a detectable antigen/antibody level).

Due to its complexity, NAA testing has traditionally been done in laboratory settings. That is, a patient visits a doctor's office or a laboratory to provide the biological sample which is processed through complex and expensive machines. The processing generally involves polymerase chain reaction (PCR) reactions to generate millions (and even billions) of copies of target sequence for optimal detection. These processes require complex equipment and specialized training.

Unlike rapid antigen testing, NAA testing has been impractical and cost prohibitive to be performed in a setting outside of a traditional laboratory. For example, the sample collection and amplification equipment involve a complex array of machines, test tubes and reagents and also requires specific technical skills. Therefore, while more diagnostically accurate, NAA testing has remained in laboratories and clinical settings, such as hospitals, and are not readily available for more widespread patient use.

As such, a significant improvement in NAA testing, particularly to support testing outside of the traditional laboratory setting, is therefore desired.

### SUMMARY OF THE INVENTION

Embodiments described herein solve the aforementioned problems and may provide other solutions as well. In an embodiment, a customizable testing cartridge and a reader box are provided. The testing cartridge can have multiple fluidic chambers connected by fluid tubes; the fluidic chambers being used to perform different parts of NAA testing. The testing cartridge has external interfaces, e.g., a snap on feature, for a user and/or the reader box to insert a chemical reagent and/or a reaction chamber. The reader box has dispensing structures, e.g., a spring-loaded dispenser (e.g., a Pez-type dispenser) or rotary dispenser (a birth control pill type dispenser) to dispense the chemical reagents into the testing cartridge. Such flexibility allows the testing cartridges to be manufactured as blanks and the reagents can be added in situ for the tests. In some embodiments, the testing cartridges can be manufactured having just primers and probes, and other reagents can be added, either by the user and/or the reading box, during the testing. Therefore, the cartridges can be easily manufactured, are generally cheaper, and are significantly more convenient as compared to the existing NAA testing equipment.

In one embodiment, a system for nucleic acid amplification testing is provided. The system may include a customizable testing cartridge comprising a plurality of fluidic chambers interconnected by fluidic tubes, the customizable testing cartridge having a primer and a probe and configured to receive a sample to be tested. The system may also include a reader box configured to: receive the customizable testing cartridge, insert an enzyme or other applicable reagents to the customizable testing cartridge, and generate a result of the nucleic acid amplification testing on the sample.

In another embodiment, a customizable cartridge for nucleic acid amplification testing is provided. The customizable cartridge may include a plurality of fluidic chambers interconnected by fluidic tubes, the customizable testing cartridge having a primer and a probe for the nucleic acid amplification testing in one of the plurality of fluidic chambers.

In yet another embodiment, a method of nucleic acid amplification testing is provided. The method may include inserting, to a reader box, a customizable testing cartridge comprising a plurality of fluidic chambers interconnected by fluidic tubes, the customizable testing cartridge having a primer and a probe in addition to a sample to be tested. The method may further include inserting, by the reader box, an enzyme or other applicable reagents to the customizable testing cartridge. The method may also include generating, by the reader box, a result of the nucleic acid amplification testing on the sample.

### BRIEF DESCRIPTION OF DRAWINGS

FIG. 1 depicts an example test environment, based on the example embodiments of this disclosure.

FIG. 2A depicts a cross section of an example customizable testing cartridge, according to example embodiments of this disclosure.

FIG. 2B depicts cross section of another example customizable testing cartridge, according to example embodiments of this disclosure.

FIG. 3 shows an example rotary dispenser to dispense a reagent to the testing cartridge, according to example embodiments of the present disclosure.

FIG. 4 shows an example spring-loaded dispenser to dispense a reagent to the testing cartridge, according to example embodiments of the present disclosure.

FIGS. 5A-5B show an example snap on attachment to the testing cartridge, according to example embodiments of this disclosure.

It should be understood that the above Figures are for illustrative purposes only, and therefore should not be considered limiting.

### DETAILED DESCRIPTION OF THE INVENTION

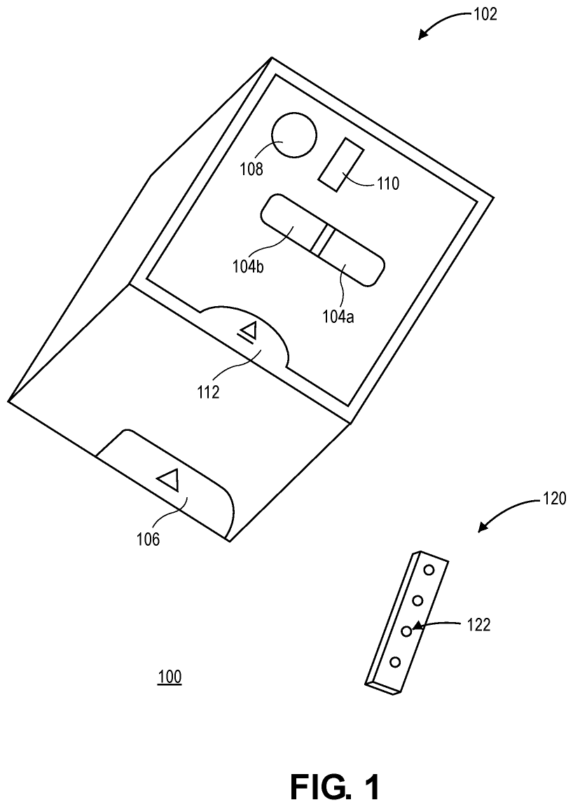

FIG. 1 depicts an example test environment 100, based on the example embodiments of this disclosure. As shown, example the test environment 100 includes a reader box 102 and a customizable testing cartridge 120. It should however be understood that the example test environment 100—with the reader box 102 and the testing cartridge 120—is just for illustration and should not be considered limiting. Alternate testing environment with alternate, additional, or fewer number of components should be considered within the scope of this disclosure.

The reader box 102, shown in a perspective view in FIG. 1, is configured to receive the testing cartridge 120 to perform a NAA test on the sample within the testing cartridge 120. To that end, the reader box 102 has one or more cartridge ports 104a, 104b (commonly referred to as a cartridge port 104 and collectively referred to as cartridge ports 104). In this example, two cartridge ports 104a, 104b are shown, where each of the cartridge ports 104a, 104b is configured to receive its own cartridge. In other examples, however, the reader box 102 can include a single cartridge port 104 or multiple cartridge ports 104, so any number of cartridge ports 104 within the reader box 102 should be considered within the scope of this disclosure.

The reader box 102 further includes a tray 106 configured to receive bulk reagents for NAA testing. The bulk reagents can include, for example but not limited to, primers, probes, and/or any other chemical reagents used for NAA testing. Other chemical reagents may include, among others, enzymes and salts. In some embodiments, the tray 106 is configured to be slid out of the reader box 102 to the load the bulk reagents. These bulk reagents are introduced into the testing cartridge 120 when loaded into the reader box 102. For example, a set of microfluidic pumps within the reader box 102 may transport the bulk reagents from the tray 106 to the testing cartridge 102. Additionally or alternatively, the reader box 102 may include a port 108 to insert a rotary dispenser (e.g., a birth control pill type dispenser). The port 108 may include any kind of attachment and/or connection mechanism to the rotary dispenser. For example, the port 108 may include a circular opening to receive the rotatory dispenser, a spring-loaded mechanism to hold the rotary dispenser under mechanical tension and to move the rotary dispenser a step after an enzyme has been dispensed. The reader box 102 may also include another port 110 to insert a spring-loaded dispenser. Each of the rotary dispenser and the spring-loaded dispenser is configured to dispense a pellet, powder or lyophilized material, or the like of a chemical (e.g., enzyme) to the testing cartridge 120 when it is inserted in the reader box 102.

The reader box 102 additionally includes an eject button 112 to remove one or more inserted testing cartridges (e.g., testing cartridge 120). Generally, the eject button 112 disengages an electromechanical mechanism that engages the testing cartridge 120 to the reader box 102, thereby allowing a safe removal of the testing cartridge 120 from the reader box 102.

The reader box 102 includes additional electronic components (not shown). For example, the reader box 102 may include a processor such as, but not limited to, a microprocessor and/or a controller to control the overall functionality of the reader box 102. The reader box 102 may further include communication components, such as, but not limited to, a Bluetooth/Wi-Fi chip, for the reader box 102 to communicate with other devices, e.g., a smartphone in the vicinity or a remote server. Therefore, any form of hardware architecture and software configured to provide processing and communication functionality should be considered within the scope of this disclosure.

The reader box 102 also includes reading/testing components. For example, the reader box 102 may include a Fluorescence plate reader and/or any other similar reader components.

The testing cartridge 120 may include fluidic chambers and fluidic channels therein between (discussed in detail below). The fluidic chambers are configured to receive biological samples and/or reagents used for the NAA testing. The testing cartridge 102 may also include one or more interfaces 122 to receive biological sample and/or the reagents (also discussed in detail below).

In a non-limiting example operation, a patient provides a biological sample, including but not limited to, a nasal, oral or throat swab onto the testing cartridge 120 and loads it into a cartridge port 104 of the reader box 102. The reader box is pre-loaded with bulk reagents, for example but not limited to, using the tray 106; and with other reagents in pellet, powder, lyophilized material or comparable form, for example but not limited to, through one or more of the ports 108, 110. Alternatively, an operator (who may or may not be the patient) may attach one or more reagents or reaction chambers onto the testing cartridge 120 before loading it to the reader box 102. The reader box 102 in turn uses the reagents on the biological sample to perform the several steps of NAA testing. The results of the testing may then be provided to a smartphone and/or to a remote server for access through the Internet.

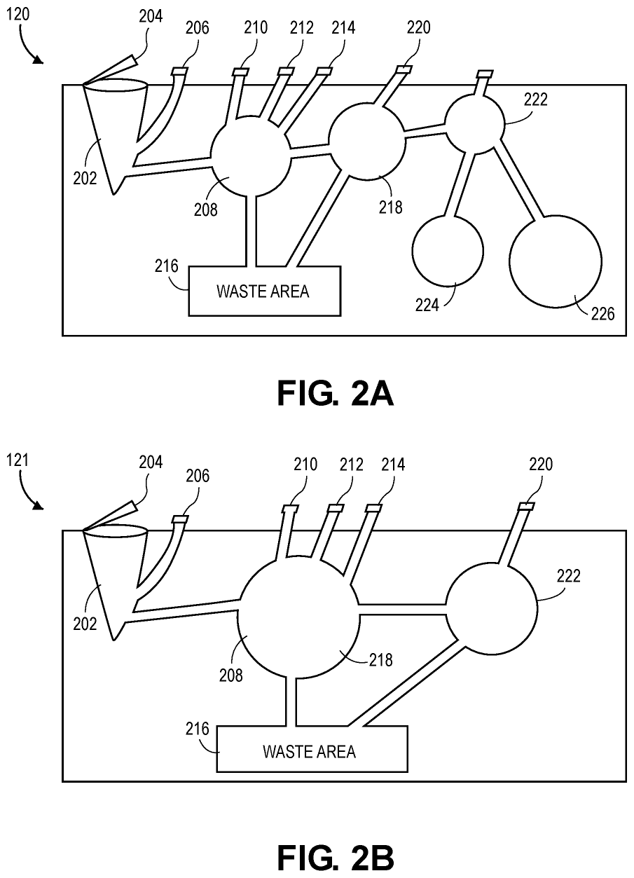

FIG. 2A depicts a cross section of the example testing cartridge 120, according to example embodiments of this disclosure. It should be understood that the shown cross section is just an illustration and should not be considered limiting. That is, cartridges with different cross section, with additional, alternative, or fewer number of components, should be considered within the scope of this disclosure. Furthermore, the structure of the testing cartridge 120 is described in terms of its functionality for an ease of understanding, and the described structure-functionality coupling should not be considered limiting.

The testing cartridge 120 includes a sample collection vial 202 with a lid 204. The lid 204 may be formed of any type of material such as plastic. The sample collection vial 202 is configured to receive the biological sample that is to be amplified through NAA and then tested. The biological sample is provided by a patient, and in some embodiments, that is all that is required of the patient and the reader box 102 box can handle all of the reagents. The biological sample may include, for example, but not limited to, sputum, saliva, blood, serum, urine, mucus, skin cells, tissue, nasal or throat swab, fecal matter, and/or any type of biological sample. The biological sample may be introduced to the sample collection vial 202 through any kind of mechanism such as, but not limited to, a swabbing tip, a blood collecting stick, and/or any other type of mechanism. For example, skin cells may be scraped or added into the testing cartridge 120. The sample collection vial 202 may also include a mechanism for determining adequacy of the biological sample. For example, the sample collection vial 202 may include a line up to which the biological sample is to be filled. The reader box 102 may include a complementary mechanism (e.g., an optical reader) to determine and indicate a presence of an adequate amount of biological sample. A sample buffer port 206 is configured to receive a sample buffer to keep a stable pH environment for the sample.

A lysis chamber 208 is formed within the testing cartridge 120. The sample in the sample collection vial 202 can be moved to the lysis chamber 208 through any kind of mechanism, such as, but not limited to, capillary action, gravity, pressure applied by the reader box 102, e.g., by using the sample buffer. The lysis chamber 208 may have multiple ports to receive several reagents and/or components required for the NAA testing from the reader box 102. For example, port 210 receives Guanidine Hydrocholoride (HCl) to be added to the sample. Port 212 receives isopropyl alcohol and magnetic beads from the reader box 102. Port 214 receives a wash buffer from the reader box 102.

Once all the 210, 212, 214 have received the corresponding reagents and components, the mixture in the lysis chamber 208 is heated, for example, heated for 2 minutes to 90 degrees Celsius. The heating can be performed by the reader box 102 through any kind of mechanism. For example, the reader box 102 may have a resistive heating wire that is in close proximity to the lysis chamber 208 to provide the requisite amount of heat. After the mixture is heated, the reader box 102 may turn on its magnets (e.g., electromagnets) to pull the magnetic beads to a side of the lysis chamber 208. The waste is then pushed to the waste area 216. The reader box 102 can add the wash buffer (i.e., through port 214) and push the waste to the waste area 216 for a few more cycles. After the washing cycles are completed, the suspension in the lysis chamber 208 is moved an elution chamber 218 by using the magnetic beads (e.g., through the use of electromagnets).

In the elution chamber 218 formed within the testing cartridge 120, the magnetic beads may be pulled aside and the waste may be pushed to the waste area 216. The magnetic beads are again released within the elution chamber. The reader box 102 the adds an elution buffer through port 220 to the elution chamber 218. After the addition of the elution buffer, the magnetic beads are again pushed aside and the mixture is pushed to chamber 222.

Within the chamber 222, an enzyme pellet is dropped to the mixture through a dispenser. In some embodiments, the dispenser may include a spring-loaded Pez-type dispenser that drops one enzyme pellet to the chamber 222. In some embodiments, the dispenser may include a rotary dispenser, for example but not limited to, a birth control type dispenser, that drops one or more enzyme pellets and/or other reagents into the chamber.

The mixture is then pushed to the chambers 224, 226, where primers and probes, e.g., lyophilized, may be pre-loaded. For example, chamber 226 may have primers and probes for one pathogen and the chamber 224 may have primers and probes for another pathogen. In these chambers 224, 226, an isothermal amplification may be started at 60 degrees Celsius. By way of example, a fluorescent detection can be performed at this temperature in the green, red, and any other spectrum.

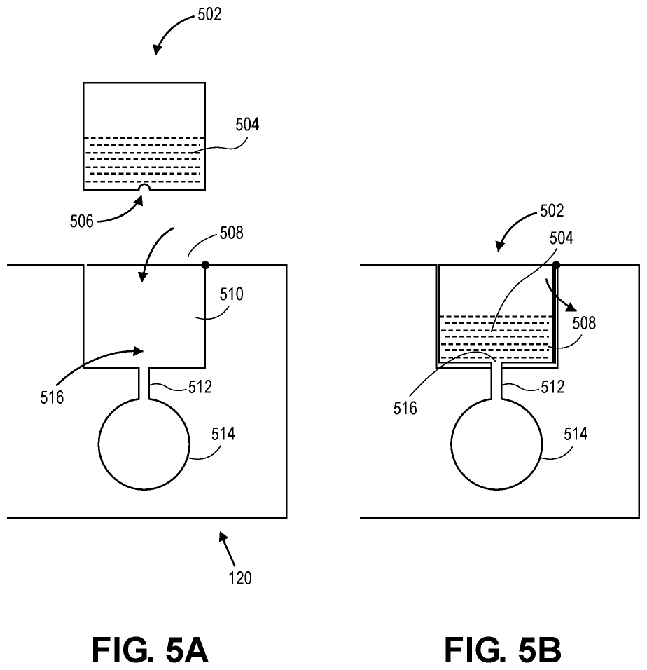

It should, however, be understood that the pre-loaded primers and the probes in the chambers 224, 226 are optional, e.g., these chambers can be blank during the time of manufacture. All of the reagents can be added either by the reader box 102 or the user. Essentially, the testing cartridge 120 can be manufactured as a blank to be rapidly customizable during its use. Optionally, the chambers 224, 226 themselves can be optional, and an alternate testing cartridge can be manufactured without the chambers 224, 226. For example, FIG. 2B depicts cross section of another example testing cartridge 121, according to example embodiments of this disclosure. Particularly, the example testing cartridge 121 may not have the chambers 224, 226 for the primers and the probes. The primers and the probes may be directly added, for example, by snap on attachments as described below in reference to FIGS. 5A-5B. The snap on attachments in turn may themselves provide the functionality of the chambers 224, 226 shown in FIG. 2A.

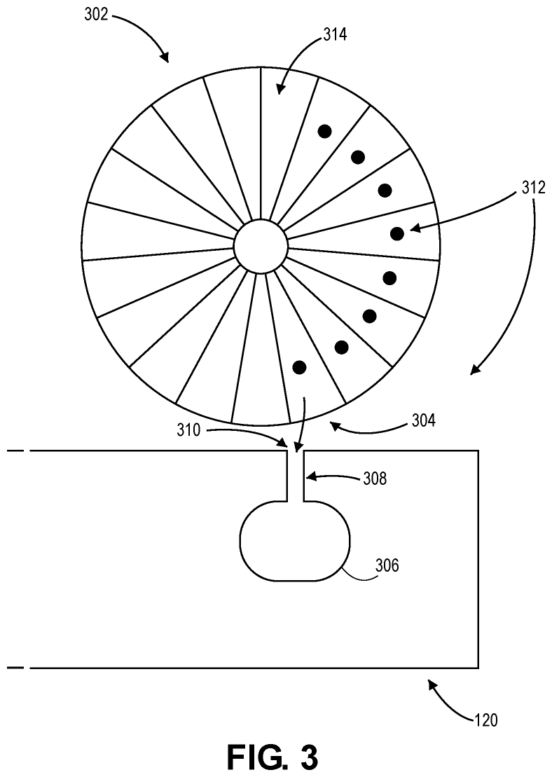

FIG. 3 shows an example rotary dispenser 302 to dispense a reagent to the testing cartridge 120, according to example embodiments of the present disclosure. The rotary dispenser 302 and its interface with the testing cartridge 120 is just intended as an illustration and should not be considered limiting.

As shown, the rotary dispenser 302 is divided into multiple chambers 314. Each chamber 314 can include a single piece of a dry or lyophilized reagent 312 (e.g., an enzyme pellet). The rotary dispenser 302 can be loaded to the reader box 102 through the port 108. The rotary dispenser 302 may be rotated (as shown, in a clockwise direction) using a motorized mechanism in the reader box 102. The rotation generally causes a chamber of the rotary dispenser 302 to interface with a port 310 of the testing cartridge 120. The chamber interfacing the port 310 may have any kind of opening mechanism 304 that allows the dry or lyophilized reagent 312 stored therein to move away from the chamber. For example, the opening mechanism 304 may a swinging door, which may open due to gravity and the dry or lyophilized reagent 312 move to the port 310 through the action of gravity itself. In other embodiments, the reader box 102 may have a counterpart mechanical/electromechanical part that engages the opening mechanism 304, which allows the dry or lyophilized reagent 312 to move down due to the action of gravity. In other embodiments, the reader box 102 can generate an air pressure to open the opening mechanism 304 and move the dry or lyophilized reagent 312 due to the action of gravity. These are just but some examples of the release of the dry or lyophilized reagent 312 and should not be considered limiting. Any kind of dry or lyophilized reagent 312 releasing mechanism should be considered within the scope of this disclosure.

Regardless of how it is released, the dry or lyophilized reagent 312 may travel to in the testing cartridge 120 through the port 310. The dry or lyophilized reagent 312 may then travel down the chute 308 to reach a reaction chamber to mix with the sample being amplified. Upon release of the dry or lyophilized reagent 312, the rotary dispenser 302 moves one position (as shown, in a clockwise direction, such that another loaded chamber 314 aligns with the port 310 of the testing cartridge 120.

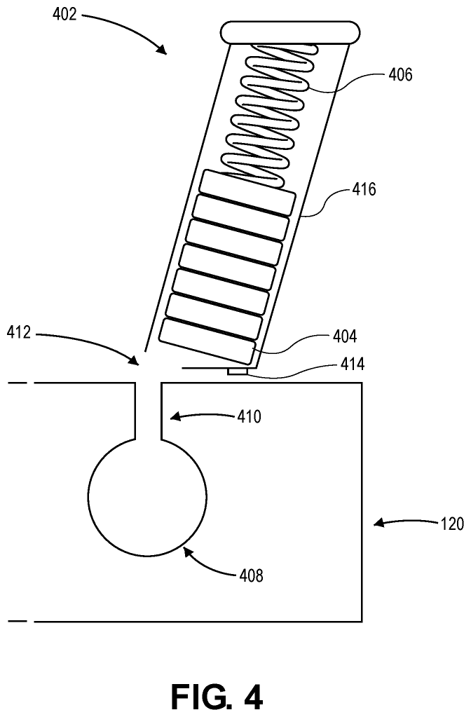

FIG. 4 shows an example spring-loaded dispenser 402 to dispense a reagent to the testing cartridge 120, according to example embodiments of the present disclosure. The spring-loaded dispenser 402 and its interface with the testing cartridge 120 is just intended as an illustration and should not be considered limiting.

The spring-loaded dispenser 402 includes a hollow body 416 where multiple dry or lyophilized reagents 404 are loaded. A spring 406 within the hollow body 416 provides an actuation mechanism to push the dry or lyophilized reagents 404 out of the hollow body and onto a port 412 of the testing cartridge 120. For example, the spring 406 provides enough force to push one dry or lyophilized reagent 404 out of the hollow chamber when the port 412 of the testing cartridge 120 aligns with a bottom area 414 of the spring-loaded dispenser 402. When one pellet of the dry or lyophilized reagent 404 is thus dispensed, the spring 406 pushes another dry or lyophilized reagent 404 at the end of the spring-loaded dispenser 402 for another test. The dispensed dry or lyophilized reagent travels down a chute 410 to reach a reaction chamber to perform a step of the NAA amplification process.

FIGS. 5A-5B show an example snap on attachment 502 to the testing cartridge 120, according to example embodiments of this disclosure. The snap on attachment 502 is attached to the testing cartridge 120 to create an additional reaction chamber within the testing cartridge 120 and/or add additional reagents within the testing cartridge 120. As with the above embodiments, it should be understood that the snap on attachment 502 is just shown for illustrative purposes only and should not be considered limiting. That is, snap on attachments with alternative structures and functionality should be considered within the scope of this disclosure.

As shown, the snap on attachment 502 includes a reagent 504 within its walls. The snap on attachment 502 may further contain a fungible part 506 that may be pierced and/or otherwise destroyed for other components of the testing cartridge 120 to access the snap on attachment 502 when it is attached to the testing cartridge 120.

The counterpart section of the testing cartridge 120 includes a receiving chamber 510 that is configured to receive the snap on attachment 502. A trapdoor 508 that swivels inside the receiving chamber 510 is provide on top of the receiving chamber 510. A fluidic tube 512 with a piercing member 516 interfaces the receiving chamber 510.

To attach the snap on attachment 502, a user may press the snap on attachment 502 against the trapdoor 508, which in turn will swivel inward. Upon further application of the pressure, the snap on attachment 502 goes inside the receiving chamber 510. The piercing member 516 pierces the fungible part 506 thereby allowing the fluidic tube 512 and a reaction chamber 514 to access the reagent 504 within the snap on attachment 502. The reagent 504 may include, but is not limited to dry, lyophilized, or liquid reagent.

The reagent 504 generally includes primers and probes for specific NAA testing. For example, different snap on attachments 502 can be developed for the different NAA testing. Therefore, a blank testing cartridge 120 (e.g., having magnetic beads but no reagents) can be rapidly customized at the time of testing using the snap on attachments 502 with specific primers and probes for the desired NAA tests. In some embodiments, the same testing cartridge 120 may have multiple receiving chambers 510 for multiple snap on attachments 502. For example, testing cartridge 120 can have three snap on attachments 502—with three different types of primers and probes—for three different NAA tests.

NAA testing as described herein differs from other tests in that they detect genetic materials (RNA or DNA) rather than antigens or antibodies. Detection of genetic material allows an early diagnosis of a disease because the detection of antigens and/or antibodies often requires time for them to start appearing in the bloodstream. Since the amount of a certain genetic material is usually very small, many NAAs include a step that amplifies the genetic material and makes many copies of it. Such NAAs are also referred to as nucleic acid amplification tests (NAATs). There are several possible techniques for amplification envisioned for use in the present invention, including but not limited to polymerase chain reaction (PCR), strand displacement assay (SDA), or transcription mediated assay (TMA). Embodiments disclosed herein may apply to isothermal nucleic acids amplification and detection reactions as well. Examples for this category of analytical methods are LAMP (loop mediated isothermal amplification), RPA (recombinase polymerase amplification), tHDA (helix dependent amplification), NEAR (nicking enzyme amplification reaction), and NASBA (nucleic acid sequence-based amplification).

The methods of the invention are useful for detection of the presence of pathogens (e.g., viral, such as coronavirus, or bacterial) and also for early detection of cancer or other diseases, e.g., through genetic mutations. For example, cancerous cells may have specific biomarkers (showing the corresponding genetic mutations) in their nucleic acid materials, which may be amplified by the techniques disclosed herein. The amplified biomarkers may then be used to detect the specific type of cancer. Some non-limiting examples of the cancer include liver cancer, brain cancer, blood cancer, bone cancer, skin cancer, and/or any other type of cancer. Nucleic acid used in the device and methods of invention include human and non-human (e.g., veterinary) samples. In addition, the invention is useful for forensic purposes when the amount of nucleic acid in a sample is lower than the detection limits for typical testing. It should be understood that DNA, RNA or both can be detected by invention methods.

The nucleic acids according to the present disclosure are not limited and include any nucleic acid. By way of example and not limitation, the nucleic acid may be: DNA, including but not limited to genomic DNA, mitochondrial DNA, bacterial DNA, viral DNA, plasmids, cosmids, linear oligodeoxynucleotides and polydeoxynucleotides, cDNA, PCR fragments, PCR amplicons, tHDA amplicons, LCR amplicons, long-range PCR amplicons, oligonucleotides, primers, probes, artificial or synthetic DNA; RNA, including but not limited to mRNA, tRNA, rRNA, viral RNA, siRNA, miRNA, RNAi, linear oligonucleotides, linear polynucleotides, probes, artificial or synthetic RNA; artificial nucleic acids such as PNA and LNA as well as combinations thereof such as nucleic acids comprising both DNA and RNA, and hybrids thereof such as RNA:DNA hybrids; complexes of nucleic acids with other biological components. The nucleic acid may be single-stranded or double-stranded. It may contain modifications such as natural modifications and artificial modifications, and may contain artificial nucleotides comprising, e.g., artificial bases, artificial sugar moieties and/or artificial connections between the nucleotides.

Nucleic acids can include, without limitation, nucleic acids found in specimens or cultures (e.g., cellular, microbial, or microbiological) including biological and environmental samples. The nucleic acids may be found in any biological samples from cell culture, bacteria, viruses, an animal, including a human, fluid, solid (e.g., stool) or tissue samples. Target nucleic acids may further be found in biological samples including, but not limited to cervical samples (e.g., a sample obtained from a cervical swab), adenoid cells, skin cells, anal epithelial cells, blood, blood products such as serum, plasma or buffy coat, saliva, cerebral spinal fluid, pleural fluid, milk, lymph, sputum, urine and semen. In other embodiments, the nucleic acids are from other virus, bacteria, parasites, for example Herpesviridae, HIV, Chlamydia trachomatis, Neisseria gonorrhoeae spp, Mycobacterium tuberculosis, Coronaviridae, and/or influenza.

In one embodiment, the nucleic acids are human papillomavirus (HPV) and include genetic variants of HPV. A variant includes polymorphisms, mutants, derivatives, modified, altered, or other forms of the nucleic acid. In one embodiment, the nucleic acid is an HPV nucleic acid. In another embodiment, the HPV nucleic acid is HPV DNA and/or RNA of a high-risk HPV type. In another embodiment, the nucleic acids are high risk HPV types such as, 16, 18, 31, 33, 35, 39, 45, 51, 52, 56, 58, 59, 68, 26, 66, and/or 82.

Samples that contain nucleic acid include, but are not limited to, a specimen or culture (e.g., cellular, microbiological and viral cultures) including biological and environmental samples. Biological samples may be from any source such as cell culture, bacteria, viruses, an animal, including a human, fluid, solid (e.g., stool) or tissue samples, as well as liquid and solid food and feed products and ingredients such as dairy items, vegetables, meat and meat byproducts, and waste. Environmental samples include, for example, environmental material such as surface matter, soil, water and industrial samples, as well as samples obtained from food and dairy processing instruments, apparatus, equipment, utensils, disposable and non-disposable items. Exemplary biological samples including, but not limited to, cell samples, such as cervical epithelial cells (e.g., a sample obtained from a cervical swab), adenoid cells, anal epithelial cells, blood, blood products such as serum, plasma or buffy coat, saliva, cerebral spinal fluid, pleural fluid, milk, lymph, sputum and semen, and may be collected, for example, in Preservcyt, Surepath and/or Digene Collection Medium (“DCM”). The sample may comprise a deoxyribonucleic acid (DNA) and/or ribonucleic acid (RNA).

In some aspects, the devices and methods can be used to detect the presence or absence of nucleic acids associated with one or more viruses in a biological sample. Non-limiting examples of virus types include double-stranded DNA viruses, single-stranded DNA viruses, double-stranded RNA viruses, or single-stranded RNA viruses. Single-stranded RNA viruses can replicate directly or can include DNA intermediates in their life cycle. DNA viruses can replicate directly or through RNA intermediates.

In some aspects, the devices and methods can be used to detect the presence or absence of nucleic acids associated with one or more fungi in a biological sample. Examples of fungal pathogens include, but are not limited to, Aspergillus, Blastomyces, Coccidioides, cryptococcus (Cryptococcus), histoplasma (Histoplasma), paracoccidioides (Paracoccidiides), sporothrix (Sporothrix), Pneumocystis, Candida spp and Zygomycetes.

In some aspects, the devices and methods can be used to detect the presence or absence of nucleic acids associated with one or more parasites in a biological sample. Non-limiting examples of parasites include Plasmodium (Plasmodium), leishmania (Leishmania), babesia (Babesia), treponema (Treponema), trypanosoma (Trypanosoma), Toxoplasma gondii (Toxoplasma gondii), plasmodial spp, trypanosoma (Trypanosoma spp.) or bacteria. That is, the embodiments disclosed herein can be used to detect any type of nucleic acid (e.g., DNA, RNA) of any type of pathogen (e.g., virus, bacteria, etc.) to detect the various example diseases disclosed herein and/or other diseases.

In some cases, the biological sample may be contained in an environmental sample containing a medium, such as water, soil, air, and the like. In some cases, the biological sample may be a forensic sample (e.g., hair, blood, bone, semen, saliva, etc.). In some cases, the biological sample may contain pathogens for bioterrorism attacks (e.g., influenza, anthrax, smallpox).

In some aspects, the biological sample comprises an infectious pathogen associated with a Sexually Transmitted Disease (STD) or a Sexually Transmitted Infection (STI). Non-limiting examples of STDs or STIs and related infectious pathogens that can be detected using the devices and methods provided herein can include Bacterial Vaginosis (Bacterial Vaginosis); chlamydia (Chlamydia) (Chlamydia trachomatis); genital herpes (Genital herpes) (herpes virus); gonorrhea (Gonorrhea) (Neisseria gonorrhoeae); Hepatitis (e.g., Hepatitis A, B, C, D, E); genital warts, anal warts, cervical cancer (Human papillomaviras); lymphogranuloma venereum (Chlamydia trachomatis)); syphilis (Syphilis) (Treponema pallidum); trichomoniasis (Trichomonas) (Trichomonas vaginalis); yeast infection (Candida spp); and acquired immunodeficiency syndrome (human immunodeficiency virus); and/or Plasmodium spp (e.g.,. Falciparum, ovale, vivax, etc.)

In one aspect of the invention, the NAA test can be performed in a home setting. The invention provides the advantage of not having to be employed in a traditional laboratory setting and is therefore useful in facilities such as by way of example, correctional facilities, military bases, ships, infirmaries (e.g., military, university), nursing homes, veterinary offices, zoos, elder care facilities, small or rural communities, patient's homes, or other facilities which may otherwise require a patient to travel to a doctor's office or hospital, for example.

Embodiments disclosed herein may be used for quantification NAA methods to detect the pathogen load (e.g., viral load, bacterial load) in a sample. That is, the number of replications may be estimated through statistical techniques and/or counted to determine the initial pathogen load in the biological sample. A larger number of replications may indicate a lower pathogen load in the biological sample and a smaller number of replications may indicate a higher pathogen load in the biological sample.

Additional examples of the presently described method and device embodiments are suggested according to the structures and techniques described herein. Other non-limiting examples may be configured to operate separately or can be combined in any permutation or combination with any one or more of the other examples provided above or throughout the present disclosure.

It will be appreciated by those skilled in the art that the present disclosure can be embodied in other specific forms without departing from the spirit or essential characteristics thereof. The presently disclosed embodiments are therefore considered in all respects to be illustrative and not restricted. The scope of the disclosure is indicated by the appended claims rather than the foregoing description and all changes that come within the meaning and range and equivalence thereof are intended to be embraced therein.

It should be noted that the terms “including” and “comprising” should be interpreted as meaning “including, but not limited to”. If not already set forth explicitly in the claims, the term “a” should be interpreted as “at least one” and “the”, “said”, etc. should be interpreted as “the at least one”, “said at least one”, etc. Furthermore, it is the Applicant's intent that only claims that include the express language “means for” or “step for” be interpreted under 35 U.S.C. 112(f). Claims that do not expressly include the phrase “means for” or “step for” are not to be interpreted under 35 U.S.C. 112(f).

Although the invention has been described with reference to the presently preferred embodiment, it should be understood that various modifications can be made without departing from the spirit of the invention. Accordingly, the invention is limited only by the following claims.

What is claimed is:

1. A system for nucleic acid amplification testing, comprising:
a customizable testing cartridge comprising a plurality of fluidic chambers interconnected by fluidic tubes, the customizable testing cartridge having a primer and a probe and configured to receive a sample to be tested; and
a reader box configured to:
receive the customizable testing cartridge,
insert an enzyme or reagent to the customizable testing cartridge, and
generate a result of the nucleic acid amplification testing on the sample.

2. The system of claim 1, wherein the primer and the probe are pre-loaded on the customizable testing cartridge during manufacturing of the customizable testing cartridge.

3. The system of claim 1, wherein the primer and the probe are attached as snap on attachments to the customizable testing cartridge before its insertion in the reader box.

4. The system of claim 1, wherein the reader box comprises a spring-loaded enzyme dispenser configured to dispense the enzyme to the customizable testing cartridge.

5. The system of claim 1, wherein the reader box comprises a rotary enzyme dispenser configured to dispense the enzyme to the customizable testing cartridge.

6. The system of claim 1, wherein the reader box comprises a tray of reagents configured to be inserted into the customizable testing cartridge through a pumping mechanism.

7. The system of claim 1, wherein the primer and the probe are configured for a first nucleic acid amplification testing, the customizable testing cartridge comprising a second primer and a second probe for a second nucleic acid amplification testing.

8. The system of claim 1, wherein the reader box is configured to:
receive a second customizable testing cartridge,
insert a second enzyme or reagent to the customizable testing cartridge, and
generate a second result of a second nucleic acid amplification testing, wherein the nucleic acid amplification testing and the second nucleic acid amplification testing are performed in parallel.

9. The system of claim 1, wherein the reader box is configured to insert the enzyme as a pellet or lyophilized reagent to the customizable testing cartridge.

10. A customizable cartridge for nucleic acid amplification testing, comprising:
a plurality of fluidic chambers interconnected by fluidic tubes, the customizable testing cartridge having a primer and a probe for the nucleic acid amplification testing in one of the plurality of fluidic chambers.

11. The customizable cartridge of claim 10, wherein the primer and the probe are pre-loaded on the customizable testing cartridge during a manufacturing of the customizable testing cartridge.

12. The customizable cartridge of claim 10, wherein the primer and the probe are attached as snap on attachments to the customizable testing cartridge.

13. The customizable cartridge of claim 10, wherein the primer and the probe are configured for a first nucleic acid amplification testing, the customizable testing cartridge comprising a second primer and a second probe configured for a second nucleic acid amplification testing.

14. A method of nucleic acid amplification testing, comprising:
inserting, to a reader box, a customizable testing cartridge comprising a plurality of fluidic chambers interconnected by fluidic tubes, the customizable testing cartridge having a primer and a probe in addition to a sample to be tested;
inserting, by the reader box, an enzyme or reagent to the customizable testing cartridge; and
generating, by the reader box, a result of the nucleic acid amplification testing on the sample.

15. The method of claim 14, wherein the primer and the probe are pre-loaded on the customizable testing cartridge during manufacturing of the customizable testing cartridge.

16. The method of claim 14, further comprising:
attaching the primer and the probe a snap on attachment to the customizable testing cartridge before its insertion in the reader box.

17. The method of claim 14, wherein inserting the enzyme to the customizable testing cartridge comprises:
dispensing, by a spring-loaded enzyme dispenser in the reader box, the enzyme to the customizable testing cartridge.

18. The method of claim 14, wherein inserting the enzyme to the customizable testing cartridge comprises:
dispensing, by a rotary enzyme dispenser in the reader box, the enzyme to the customizable testing cartridge.

19. The method of claim 14, further comprising:
inserting, by the reader box, one or more reagent from a reagent tray to the customizable testing cartridge through a pumping mechanism.

20. The method of claim 14, wherein the primer and the probe are for a first nucleic acid amplification testing, the customizable testing cartridge comprising a second primer and a second probe for a second nucleic acid amplification testing.

21. The method of claim 14, wherein the sample is a biological sample.

22. The method of claim 14, wherein the nucleic acid is human or microbial nucleic acid.

23. The method of claim 22, wherein the microbial nucleic acid is bacterial, a parasitic, or viral nucleic acid.
